(19) United States
(12) Patent Application Publication
(10) Pub. No.: US 2011/0287110 A1
(43) Pub. Date: Nov. 24, 2011

(54) COMBINATION CANCER TREATMENT

(76) Inventors: Mark Wesley Dewhirst, Durham, NC (US); Dennis J. Thiele, Chapel Hill, NC (US); Chelsea D. Landon, Durham, NC (US)

(21) Appl. No.: 13/092,341

(22) Filed: Apr. 22, 2011

Related U.S. Application Data
(60) Provisional application No. 61/327,352, filed on Apr. 23, 2010.
(60) Provisional application No. 61/327,958, filed on Apr. 26, 2010.

(51) Int. Cl. A61K 33/24; A61K 31/282; A61P 35/00; A61K 31/27

(52) U.S. Cl. 424/649; 514/479; 514/492

(57) ABSTRACT

Described herein are methods of inhibiting the proliferation of cancer cells and methods of treating cancer, by administering a combination of a copper chelator and a platinum-based chemotherapeutic.

### CROSS-REFERENCE TO RELATED APPLICATIONS

This application claims priority to U.S. Provisional Application No. 61/327,352, filed on Apr. 23, 2010, and to U.S. Provisional Application No. 61/327,958, filed on Apr. 26, 2010, each of which is incorporated herein by reference in its entirety.

### STATEMENT REGARDING FEDERALLY SPONSORED RESEARCH

This invention was made with government support under P01 CA042745 awarded by the National Institutes of Health (National Cancer Institute), and R01 DK074192 awarded by the National Institutes of Health (National Institute of Diabetes and Digestive and Kidney Diseases). The United States government has certain rights in the invention.

### BACKGROUND

Since the late 1970s, the chemotherapeutic cisplatin has been in clinical use for the treatment of multiple types of cancer. Cisplatin is used in the clinic today due to the high degree of toxicity to tumor cells, but with this high degree of toxicity to tumor cells comes increased toxicity to normal tissue. The toxicity to normal tissue following drug treatment is the dose limiting factor for the majority of diseases. More specific or targeted drug delivery systems have been the focus of research for decades. The main goals of these delivery systems are to decrease normal tissue toxicity and to increase drug concentration to the site of disease. The development of resistance to cisplatin is also a major issue for the continuing use of cisplatin in the clinic.

### SUMMARY

In one aspect, the disclosure provides a method of reducing the proliferation of a cancer cell, comprising contacting the cancer cell with a copper chelator and a platinum-based chemotherapeutic.

In another aspect, the disclosure provides a method of treating cancer in a subject in need of treatment, comprising co-administering to the subject a platinum-based chemotherapeutic and a copper chelator, in amounts effective to treat the cancer.

In another aspect, the disclosure provides a method of enhancing the therapeutic efficacy of a platinum-based chemotherapeutic, comprising administering a copper chelator to a subject in need thereof.

In another aspect, the disclosure provides a method of increasing the susceptibility of a cancer cell to a platinum-based chemotherapeutic, comprising contacting the cancer cell with a copper chelator.

In another aspect, the disclosure provides a method of increasing uptake of a platinum-based chemotherapeutic in a cancer cell, comprising contacting the cancer cell with a copper chelator.

In another aspect, the disclosure provides a method of treating cancer in a subject in need of treatment, comprising identifying a subject having cancer, and administering to the subject a platinum-based chemotherapeutic and a copper chelator, in amounts effective to treat the cancer.

Other aspects and embodiments are encompassed by the disclosure and will become apparent in light of the following description and drawings.

### BRIEF DESCRIPTION OF THE DRAWINGS

FIG. 1 depicts Ctr1 expression levels in bladder cancer cell lines.

FIG. 2 depicts sensitivity to cisplatin in Ctr1−/− mouse embryonic fibroblasts (MEFs).

FIG. 3 depicts cisplatin uptake/accumulation in Ctr1−/− MEFs.

FIG. 4 depicts platinum-DNA adduct formation in Ctr1−/− MEFs.

FIG. 5 depicts Ctr1 mRNA knockdowns in 5637 bladder cancer cells using siRNA.

FIG. 6 depicts platinum-DNA adduct formation in bladder cancer cells following Ctr1 knockdown using siRNA.

### DETAILED DESCRIPTION

Copper is a key nutrient for biological processes including mitochondrial respiration and free radical detoxification. Ctr1 is a copper transporter located on the cell membrane. This ATP independent transporter has a high affinity for copper and allows sufficient amounts of copper to enter the cell for normal metabolic function. In addition to its role as a copper transporter, Ctr1 can also function as a transporter of platinum-based compounds such as cisplatin. Previous studies show that deleting Ctr1 in cells resulted in decreased cisplatin uptake and increased cisplatin resistance (Ishida et al. Proc. Natl. Acad. Sci. USA. 2002, 99(22):14298-302; Larson et al. Mol. Pharmacol. 2009, 75(2):324-30).

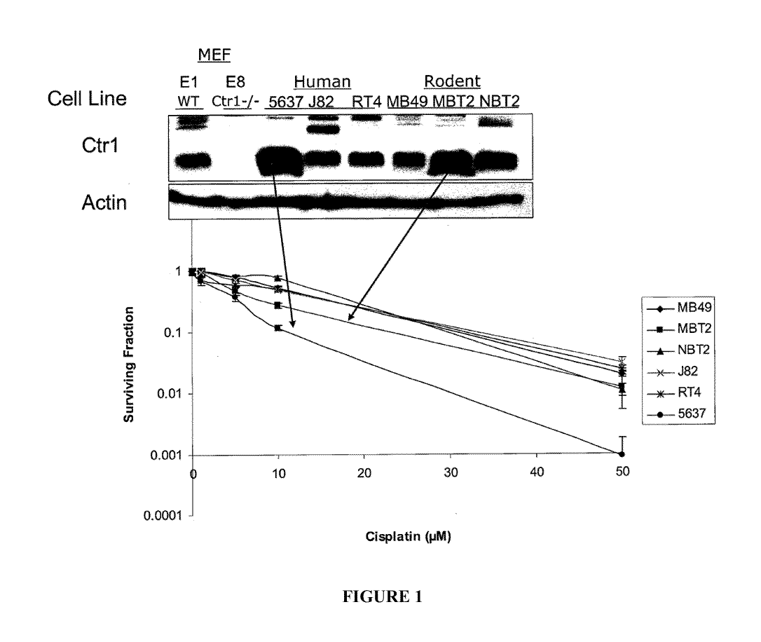

Ctr1 has been detected at the RNA level in all organs and tissues examined with high basal expression observed in the heart, liver, pancreas, prostate, colon, and intestine (Zhou et al. Proc. Natl. Acad. Sci. USA. 1997, 94(14):7481-6). As described herein, Ctr1 expression can be detected in cancer cells such as, for example, the multiple bladder cancer cell lines illustrated in the Examples. The bladder cancers have been assessed for basal Ctr1 protein levels and cells having higher Ctr1 expression are more sensitive to cisplatin (FIG. 1).

Copper chelation may create a copper-deficient environment around a cell such as, for example, a cancer cell. This copper deficiency may result in increased Ctr1 expression levels on the cell surface. Increased Ctr1 membrane expression in the presence of cisplatin may lead to increased drug uptake and accumulation and eventually tumor cell death.

Thus, in an aspect the disclosure relates to a method of reducing the proliferation of a cancer cell, comprising contacting the cancer cell with a copper chelator and a platinum-based chemotherapeutic.

In an aspect, the disclosure provides a method of reducing the proliferation of a cancer cell, comprising contacting the cancer cell with a copper chelator and a platinum-based chemotherapeutic.

In an aspect, the disclosure provides a method of treating cancer in a subject in need of treatment, comprising co-administering to the subject a platinum-based chemotherapeutic and a copper chelator, in amounts effective to treat the cancer.

In an aspect, the disclosure provides a method of enhancing the therapeutic efficacy of a platinum-based chemotherapeutic, comprising administering a copper chelator to a subject in need thereof.

In an aspect, the disclosure provides a method of increasing the susceptibility of a cancer cell to a platinum-based chemotherapeutic, comprising contacting the cancer cell with a copper chelator.

In an aspect, the disclosure provides a method of increasing uptake of a platinum-based chemotherapeutic in a cancer cell, comprising contacting the cancer cell with a copper chelator.

In an aspect, the disclosure provides a method of treating cancer in a subject in need of treatment, comprising identifying a subject having cancer, and administering to the subject a platinum-based chemotherapeutic and a copper chelator, in amounts effective to treat the cancer.

### DEFINITIONS

“Administration” or “administering,” as used herein, refers to providing, contacting, and/or delivery of a compound or compounds by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional or intracranial injection), transdermal, topical, buccal, rectal, vaginal, nasal, ophthalmic, via inhalation, and implants.

“Co-administered,” as used herein, refers to simultaneous or sequential administration of multiple compounds or agents. A first compound or agent may be administered before, concurrently with, or after administration of a second compound or agent. The first compound or agent and the second compound or agent may be simultaneously or sequentially administered on the same day, or may be sequentially administered within 1 day, 2 days, 3 days, 4 days, 5 days, 6 days, 1 week, 2 weeks, 3 weeks or one month of each other. Suitably, compounds or agents are co-administered during the period in which each of the compounds or agents are exerting at least some physiological effect and/or has remaining efficacy.

“Contacting,” as used herein as in “contacting a cell,” refers to contacting a cell directly or indirectly in vitro, ex vivo, or in vivo (i.e. within a subject, such as a mammal, including humans, mice, rats, rabbits, cats, and dogs). Contacting a cell, which also includes “reacting” a cell, can occur as a result of administration to a subject. Contacting encompasses administration to a cell, tissue, mammal, subject, patient, or human. Further, contacting a cell includes adding an agent to a cell culture. Other suitable methods may include introducing or administering an agent to a cell, tissue, mammal, subject, or patient using appropriate procedures and routes of administration as defined herein.

“Ctr1” refers to a membrane associated, homotrimeric protein that transports reduced copper (Cu(I)) in to cells. As used herein, the term Ctr1 encompasses any ortholog, variant, or functional fragment thereof. Ctr1 can include, for example, the sequence described in GenBank Accession No. NP_{—}001850. When the methods described herein benefit from the detection of Ctr1 in a cell, Ctr1 expression can be evaluated by any method known in the art, including methods for detecting polynucleotides or proteins.

“Effective amount,” as used herein, refers to a dosage of the compounds or compositions effective for eliciting a desired effect. This term as used herein may also refer to an amount effective at bringing about a desired in vivo effect in an animal, mammal, or human, such as reducing proliferation of a cancer cell.

“Pharmaceutically acceptable,” as used herein, pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

“Reducing proliferation of a cell,” as used herein, refers to reducing, inhibiting, or preventing the survival, growth, or differentiation of a cell, including killing a cell. A cell can be derived from any organism or tissue type and includes, for example, a cancer cell (e.g., neoplastic cells, tumor cells, and the like).

As used herein, the term “subject” is intended to include human and non-human animals. Exemplary human subjects include a human patient having a disorder, e.g., a disorder described herein, or a normal subject. The term “non-human animals” includes all vertebrates, e.g., non-mammals (such as chickens, amphibians, reptiles) and mammals, such as non-human primates, domesticated and/or agriculturally useful animals (such as sheep, dogs, cats, cows, pigs, etc.), and rodents (such as mice, rats, hamsters, guinea pigs, etc.).

“Susceptibility,” as used herein regarding a cancer cell, refers to the degree to which a cancer cell is affected by a chemotherapeutic agent. The cancer cell may not be affected at all, it may have its growth or proliferation slowed or halted without its being killed, or it may be killed. Susceptibility also refers to the degree a population of cancer cells, such as a tumor, is affected by a chemotherapeutic agent. “Increasing the susceptibility” of a cancer cell to a chemotherapeutic following contact or treatment with an agent, e.g., a copper chelator, indicates that the cell is more affected by the chemotherapeutic agent than a corresponding cancer cell that has not been exposed to the agent.

As used herein, the term “treat” or “treating” a subject having a disorder refers to administering a regimen to the subject, e.g., the administration of a combination of a copper chelator and a platinum-based therapeutic, such that at least one symptom of the disorder is cured, healed, alleviated, relieved, altered, remedied, ameliorated, or improved. Treating includes administering an amount effective to alleviate, relieve, alter, remedy, ameliorate, improve or affect the disorder or the symptoms of the disorder. The treatment may inhibit deterioration or worsening of a symptom of a disorder.

### Platinum-Based Chemotherapeutics

Platinum-based chemotherapeutic agents have been described as “the most important group of agents now in use for cancer treatment,” and are typified by cisplatin [cis-diamminedichloroplatinum (II)] (Reed, 1993, in Cancer, Principles and Practice of Oncology, pp. 390-4001) These agents, used alone or as a part of combination chemotherapy regimens, have been shown to be curative for testicular and ovarian cancers and beneficial for the treatment of lung, bladder, and head and neck cancers, among many others.

DNA damage is believed to be the major determinant of cisplatin cytotoxicity, though this drug also may induce other types of cellular damage. In addition to cisplatin, this group of drugs includes carboplatin and oxaliplatin, which like cisplatin are used clinically, and other platinum-containing drugs that are under development. These compounds are believed to act by the same or very similar mechanisms, so that conclusions drawn from the study of the bases of cisplatin sensitivity and resistance are expected to be valid for other platinum-containing drugs. Cisplatin is known to form adducts with DNA and to induce interstrand crosslinks. Adduct formation, through an as yet unknown signaling mechanism, is believed to activate some presently unknown cellular enzymes involved in programmed cell death (apoptosis), the process which is believed to be ultimately responsible for cisplatin cytotoxicity (see Eastman, 1990, Cancer Cells 2: 275-2802).

Embodiments of the methods described herein provide platinum coordination complexes wherein platinum is in the Pt(II) oxidation state. Some embodiments provide platinum coordination complexes having a square planar geometry with respect to the platinum atom.

Some platinum-based chemotherapeutics may include without limitation: cisplatin, carboplatin, oxaliplatin, iproplatin, tetraplatin, lobaplatin, dicycloplatin (DCP), PLD-147, JM118, JM216, JM335, and satraplatin. Such platinum-based chemotherapeutic agents also include the platinum complexes disclosed in EP 0147926, U.S. Pat. No. 5,072,011, U.S. Pat. Nos. 5,244,919, 5,519,155, 6,503,943 (LA-12/PLD-147), 6350737, and WO 01/064696 (DCP).

### Copper Chelators

A copper chelator may be an agent capable of creating a copper deficient environment, e.g., around a cancer cell or a tumor. A copper deficient environment may increase levels of surface Ctr1, resulting in increased cellular cisplatin uptake and reduced proliferation of a cancer cell.

Mutations in copper transporters such as in Wilson disease (export pump ATP7B) result in copper accumulation in the tissues and copper toxicity in several major organ systems (Schilsky M L. Biochimie. 2009, 91(10): 1278-81). Copper chelation is necessary in patients with these diseases to reduce copper levels and toxicity. Accordingly, several copper chelators are approved for use in these patients, and may be used in the methods described herein to reduce copper levels.

Embodiments of the methods described herein provide for a copper chelator that binds copper in the Cu(I) or Cu(II) oxidation state. Some embodiments provide for a copper chelator having a higher binding affinity for Cu(I) relative to Cu(II). Some embodiments provide for a copper chelator having a higher binding affinity for Cu(II) relative to Cu(I). Copper chelators may include without limitation: penicillamine (Cuprimine®, Depen®), trientine hydrochloride (also known as triethylenetetramine hydrochloride, or Syprine®), dimercaprol, diethyldithiocarbamate (e.g., sodium diethyldithiocarbamate), bathocuproine sulfonate, and tetrathiomolybdate (e.g., ammonium tetrathiomolybdate). In some embodiments, the copper chelator is not tetrathiomolybdate. Suitably, a copper chelator may not have appreciable binding affinity for a platinum-based chemotherapeutic agent.

Tetrathiomolybdate, such as ammonium tetrathiomolybdate, may serve to chelate copper and may also compete with copper for intestinal absorption. Other agents used to control copper levels in patients with Wilson disease include zinc salts, such as zinc acetate (Galzin®), which also compete with copper for intestinal absorption. Zinc may also induce production of metallothionein, a protein that binds copper and prevents its transfer into the bloodstream. Accordingly, tetrathiomolybdate and/or zinc may also be used to reduce copper absorption in the methods described herein.

### Formulations

While the copper chelator and platinum-based chemotherapeutic may be administered alone in the methods described herein, they may also be presented as one or more pharmaceutical compositions (e.g., formulations). The copper chelator and the platinum-based chemotherapeutic may be formulated as separate pharmaceutical compositions, or together in a single composition. Suitably, the copper chelator and the platinum-based chemotherapeutic are formulated as separate pharmaceutical compositions. In each composition the copper chelator and/or platinum based chemotherapeutic may be formulated with one or more pharmaceutically acceptable carriers, adjuvants, excipients, diluents, fillers, buffers, stabilizers, preservatives, lubricants, or other materials well known to those skilled in the art and optionally other therapeutic or prophylactic agents.

Accordingly, the methods described herein include administration of one or more pharmaceutical compositions, as discussed herein, in which a copper chelator and/or platinum based chemotherapeutic is admixed together with one or more pharmaceutically acceptable carriers, excipients, buffers, adjuvants, stabilizers, or other materials, as described herein.

Suitable carriers, excipients, etc. can be found in standard pharmaceutical texts, for example, Remington's Pharmaceutical Sciences, 18th edition, Mack Publishing Company, Easton, Pa., 1990.

The formulations may conveniently be presented in unit dosage form and may be prepared by any methods known in the art of pharmacy. Such methods include the step of bringing into association the active compound(s) with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, lozenges, granules, powders, capsules, cachets, pills, ampoules, suppositories, pessaries, ointments, gels, pastes, creams, sprays, mists, foams, lotions, oils, boluses, electuaries, or aerosols.

Formulations suitable for oral administration (e.g. by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g., compression or molding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g. sodium lauryl sulfate); and preservatives (e.g. methyl p-hydroxybenzoate, propyl p-hydroxybenzoate, sorbic acid). Molded tablets may be made by molding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for parenteral administration (e.g. by injection, including cutaneous, subcutaneous, intramuscular, intravenous and intradermal), include aqueous and nonaqueous isotonic, pyrogen-free, sterile injection solutions which may contain anti-oxidants, buffers, preservatives, stabilizers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

Formulations suitable for topical administration (e.g. transdermal, intranasal, ocular, buccal, and sublingual) may be formulated as an ointment, cream, suspension, lotion, powder, solution, past, gel, spray, aerosol, or oil. Alternatively, a formulation may comprise a patch or a dressing such as a bandage or adhesive plaster impregnated with active compounds and optionally one or more excipients or diluents.

Formulations suitable for topical administration in the mouth include lozenges comprising the active compound in a flavored basis, usually sucrose and acacia or tragacanth; pastilles comprising the active compound in an inert basis such as gelatin and glycerin, or sucrose and acacia; and mouthwashes comprising the active compound in a suitable liquid carrier.

Formulations suitable for topical administration to the eye also include eye drops wherein the active compound is dissolved or suspended in a suitable carrier, especially an aqueous solvent for the active compound.

Formulations suitable for nasal administration, wherein the carrier is a solid, include a coarse powder having a particle size, for example, in the range of about 20 to about 500 microns which is administered in the manner in which snuff is taken, i.e. by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the active compound.

Formulations suitable for administration by inhalation include those presented as an aerosol spray from a pressurized pack, with the use of a suitable propellant, such as dichlorodifluoromethane, trichlorofluoromethane, dichoro-tetrafluoroethane, carbon dioxide, or other suitable gases. Further formulations suitable for inhalation include those presented as a nebulizer.

Formulations suitable for topical administration via the skin include ointments, creams, and emulsions. When formulated in an ointment, the active compound may optionally be employed with either a paraffinic or a water-miscible ointment base. Alternatively, the active compounds may be formulated in a cream with an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

When formulated as a topical emulsion, the oily phase may optionally comprise merely an emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Suitable emulgents and emulsion stabilizers include Tween 60, Span 80, cetostearyl alcohol, myristyl alcohol, glyceryl monostearate and sodium lauryl sulfate. The choice of suitable oils or fats for the formulation is based on achieving the desired cosmetic properties, since the solubility of the active compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as diisoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active compound, such carriers as are known in the art to be appropriate.

### Dosages

It will be appreciated that appropriate dosages of the active compounds, and compositions comprising the active compounds, can vary from patient to patient. Determining the optimal dosage will generally involve the balancing of the level of therapeutic benefit against any risk or deleterious side effects of the treatments described herein. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g. in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In general, a suitable dose of the active compound is in the range of about 100 μg to about 250 mg per kilogram body weight of the subject per day.

A suitable dose of a platinum-based therapeutic may be a standard dose. For example, a standard dosage of cisplatin for the treatment of testicular cancer is 20 mg/m² IV daily for 5 consecutive days every 3 weeks for 3 or 4 courses of therapy. A standard dosage of cisplatin for the treatment of advanced ovarian carcinoma is 30-120 mg/m² IV once every 3-4 weeks (e.g., 50-100 mg/m² IV once every 3 weeks, e.g., 100 mg/m² IV once every 4 weeks) when cisplatin is used as a single agent; 75 mg/m² IV once every 3 weeks in combination therapy with paclitaxel; or 50-100 mg/m² IV once every 3-4 weeks when used in combination with cyclophosphamide. For the treatment of advanced bladder cancer, a standard dosage of cisplatin is 50-70 mg/m² IV once every 3-4 weeks. A standard dosage for the treatment of recurrent or advanced head and neck cancer is 80-120 mg/m² IV once every 3 weeks or 50 mg/m² IV on the first and eighth days of every 4 weeks, when cisplatin is used as a single agent; when used in combination chemotherapy regimens, a standard dose is 50-120 mg/m² IV, with the frequency of administration depending on the specific regimen employed. A standard dosage of cisplatin for the treatment of cervical cancer, e.g., invasive cervical cancer, is 40-75 mg/m² have been given concurrently with radiation therapy, in weekly or daily infusions of cisplatin; when used in combination chemotherapy regimens (e.g., cisplatin and fluorouracil) for the treatment of invasive cervical cancer, cisplatin 50-75 mg/m² has been administered IV concurrently with radiation therapy. For the treatment of metastatic or recurrent cervical carcinoma, a standard dosage of cisplatin used alone or in combination therapy is 50 mg/m² IV once every 3 weeks up to a maximum of 6 courses. For the treatment of non-small cell lung carcinoma, a standard dosage of cisplatin in combination therapy is 75-100 mg/m² IV once every 3-4 weeks, depending on the specific regimen used. For the treatment of advanced esophageal cancer, a standard dosage of cisplatin 50-120 mg/m² IV once every 3-4 weeks; in combination chemotherapy regimens, a standard dosage cisplatin is 75-100 mg/m² IV once every 3-4 weeks.

A standard dosage of oxaliplatin for the treatment of advanced colorectal cancer is 85 mg/m² IV infusion and leucovorin 200 mg/m² IV infusion in dextrose 5% in water, both given over 120 min at the same time in separate bags using a Y-line, followed by 5-fluorouracil 400 mg/m² IV bolus given over 2 to 4 min, followed by 5-fluorouracil 600 mg/m² IV infusion in dextrose 5% in water 500 mL (recommended) as a 22-h continuous infusion.

A standard dosage of carboplatin for the treatment of ovarian cancer is 360 mg/m² by intravenous injection on day 1 every 4 weeks when used as a single agent; when used in combination with cyclophosphamide, a standard dosage of carboplatin 300 mg/m² by intravenous injection on day 1 every four weeks for six cycles. A standard dosage of carboplatin for the treatment of cervical cancer, in combination with other chemotherapeutic agents as a part of the BIC regimen, is 200 mg/m² IV on day 1; the cycle is repeated every 21 days.

A standard adult dosage of penicillamine for the treatment of Wilson disease is 0.75 to 1.5 grams/day. A standard adult dosage of trientine hydrochloride for the treatment of Wilson disease is 750 to 1250 mg orally per day, in 2 to 4 equally divided doses.

In the methods described herein, a copper chelator may create a copper-deficient environment around a tumor or a tumor cell, which may lead to increased Ctr1 expression and increased uptake of a platinum-based chemotherapeutic. Accordingly, dosages of platinum-based therapeutics that are lower than standard dosages may be effective in the methods described herein.

### Cancer

The methods described herein can be used with any cancer, for example those described by the National Cancer Institute. The cancer can be a carcinoma, a sarcoma, a myeloma, a leukemia, a lymphoma or a mixed type. Exemplary cancers described by the National Cancer Institute include:

Digestive/gastrointestinal cancers such as anal cancer; bile duct cancer; extrahepatic bile duct cancer; appendix cancer; carcinoid tumor, gastrointestinal cancer; colon cancer; colorectal cancer including childhood colorectal cancer; esophageal cancer including childhood esophageal cancer; gallbladder cancer; gastric (stomach) cancer including childhood gastric (stomach) cancer; hepatocellular (liver) cancer including adult (primary) hepatocellular (liver) cancer and childhood (primary) hepatocellular (liver) cancer; pancreatic cancer including childhood pancreatic cancer; sarcoma, rhabdomyo sarcoma; islet cell pancreatic cancer; rectal cancer; and small intestine cancer;

Endocrine cancers such as islet cell carcinoma (endocrine pancreas); adrenocortical carcinoma including childhood adrenocortical carcinoma; gastrointestinal carcinoid tumor; parathyroid cancer; pheochromocytoma; pituitary tumor; thyroid cancer including childhood thyroid cancer; childhood multiple endocrine neoplasia syndrome; and childhood carcinoid tumor;

Eye cancers such as intraocular melanoma; and retinoblastoma;

Musculoskeletal cancers such as Ewing's family of tumors; osteosarcoma/malignant fibrous histiocytoma of the bone; childhood rhabdomyosarcoma; soft tissue sarcoma including adult and childhood soft tissue sarcoma; clear cell sarcoma of tendon sheaths; and uterine sarcoma;

Breast cancer such as breast cancer including childhood and male breast cancer and breast cancer in pregnancy;

Neurologic cancers such as childhood brain stemglioma; brain tumor; childhood cerebellar astrocytoma; childhood cerebral astrocytoma/malignant glioma; childhood ependymoma; childhood medulloblastoma; childhood pineal and supratentorial primitive neuroectodermal tumors; childhood visual pathway and hypothalamic glioma; other childhood brain cancers; adrenocortical carcinoma; central nervous system lymphoma, primary; childhood cerebellar astrocytoma; neuroblastoma; craniopharyngioma; spinal cord tumors; central nervous system atypical teratoid/rhabdoid tumor; central nervous system embryonal tumors; and childhood supratentorial primitive neuroectodermal tumors and pituitary tumor;

Genitourinary cancers such as bladder cancer including childhood bladder cancer; renal cell (kidney) cancer; ovarian cancer including childhood ovarian cancer; ovarian epithelial cancer; ovarian low malignant potential tumor; penile cancer; prostate cancer; renal cell cancer including childhood renal cell cancer; renal pelvis and ureter, transitional cell cancer; testicular cancer; urethral cancer; vaginal cancer; vulvar cancer; cervical cancer; Wilms tumor and other childhood kidney tumors; endometrial cancer; and gestational trophoblastic tumor; Germ cell cancers such as childhood extracranial germ cell tumor; extragonadal germ cell tumor; ovarian germ cell tumor;

Head and neck cancers such as lip and oral cavity cancer; oral cancer including childhood oral cancer; hypopharyngeal cancer; laryngeal cancer including childhood laryngeal cancer; metastatic squamous neck cancer with occult primary; mouth cancer; nasal cavity and paranasal sinus cancer; nasopharyngeal cancer including childhood nasopharyngeal cancer; oropharyngeal cancer; parathyroid cancer; pharyngeal cancer; salivary gland cancer including childhood salivary gland cancer; throat cancer; and thyroid cancer;

Hematologic/blood cell cancers such as a leukemia (e.g., acute lymphoblastic leukemia including adult and childhood acute lymphoblastic leukemia; acute myeloid leukemia including adult and childhood acute myeloid leukemia; chronic lymphocytic leukemia; chronic myelogenous leukemia; and hairy cell leukemia); a lymphoma (e.g., AIDS-related lymphoma; cutaneous T-cell lymphoma; Hodgkin's lymphoma including adult and childhood Hodgkin's lymphoma and Hodgkin's lymphoma during pregnancy; non-Hodgkin's lymphoma including adult and childhood non-Hodgkin's lymphoma and non-Hodgkin's lymphoma during pregnancy; mycosis fungoides; Sezary syndrome; Waldenstrom's macroglobulinemia; and primary central nervous system lymphoma); and other hematologic cancers (e.g., chronic myeloproliferative disorders; multiple myeloma/plasma cell neoplasm; myelodysplastic syndromes; and myelodysplastic/myeloproliferative disorders);

Lung cancer such as non-small cell lung cancer; and small cell lung cancer;

Respiratory cancers such as adult malignant mesothelioma; childhood malignant mesothelioma; malignant thymoma; childhood thymoma; thymic carcinoma; bronchial adenomas/carcinoids including childhood bronchial adenomas/carcinoids; pleuropulmonary blastoma; non-small cell lung cancer; and small cell lung cancer;

Skin cancers such as Kaposi's sarcoma; Merkel cell carcinoma; melanoma; and childhood skin cancer;

AIDS-related malignancies;

Other childhood cancers, unusual cancers of childhood and cancers of unknown primary site;

and metastases of the aforementioned cancers can also be treated or prevented in accordance with the methods described herein.

The methods described herein may be suited to treat bladder, testicular, ovarian, head and neck, cervical, lung, mesothelioma, esophageal, melanoma, brain tumor, neuroblastoma, colorectal, Wilms' tumor, retinoblastoma, breast, endometrial, adrenocortical, anal, biliary tract, carcinoid tumors, choriocarcinoma, gastric, liver cancer, non-Hodgkin's lymphoma, osteosarcoma, soft-tissue sarcomas, penile, malignant thymoma, anaplastic thyroid cancer, rhabdoid tumor of the kidney, advanced medullary thyroid cancer, carcinoid, mesothelioma, bone, gliomas or prostate cancers. In embodiments, the methods suitably treat bladder cancer (e.g., muscle-invasive bladder carcinoma, advanced or metastatic bladder carcinoma), testicular cancer (e.g., nonseminomatous testicular carcinoma, disseminated seminoma testis or extragonadal germ-cell tumors), ovarian cancer (e.g., ovarian epithelial cancer or ovarian germ-cell tumors), head and neck cancer (e.g., squamous cell carcinoma), cervical cancer (e.g., invasive, metastatic or recurrent cervical cancer), lung cancer (e.g., small cell lung cancer or non-small cell lung cancer), Wilms' tumor, brain tumors (e.g., gliomas, medulloblastoma or germ cell tumors), neuroblastoma, retinoblastoma, mesothelioma (e.g., malignant pleural mesothelioma), esophageal cancer (e.g., localized or advanced esophageal cancer), and colorectal cancer.

### Cancer Combination Therapy

Co-administration of a copper chelator and a platinum-based chemotherapeutic may be used in combination with other known therapies. Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The copper chelator and platinum-based chemotherapeutic and the at least one additional therapeutic agent can be administered simultaneously, in the same or in separate compositions, or sequentially. For sequential administration, the copper chelator and platinum-based chemotherapeutic can be administered first, and the additional agent can be administered subsequently, or the order of administration can be reversed.

In some embodiments, the copper chelator and platinum-based chemotherapeutic are administered in combination with other therapeutic treatment modalities, including surgery, radiation, cryosurgery, and/or thermotherapy. Such combination therapies may advantageously utilize lower dosages of the administered agent and/or other chemotherapeutic agent, thus avoiding possible toxicities or complications associated with the various therapies. The phrase “radiation” includes, but is not limited to, external-beam therapy which involves three dimensional, conformal radiation therapy where the field of radiation is designed to conform to the volume of tissue treated; interstitial-radiation therapy where seeds of radioactive compounds are implanted using ultrasound guidance; and a combination of external-beam therapy and interstitial-radiation therapy.

In some embodiments, the copper chelator and platinum-based chemotherapeutic are administered with at least one additional therapeutic agent, such as a chemotherapeutic agent. In certain embodiments, the copper chelator and platinum-based chemotherapeutic are administered in combination with one or more additional chemotherapeutic agents, e.g., with one or more chemotherapeutic agents described herein.

In some embodiments, the copper chelator and platinum-based chemotherapeutic are administered in combination with a chemotherapeutic agent. Exemplary classes of chemotherapeutic agents include, e.g., the following:

alkylating agents (including, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexylen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®).

anti-EGFR antibodies (e.g., cetuximab (Erbitux®), panitumumab (Vectibix®), and gefitinib (Iressa®)).

anti-Her-2 antibodies (e.g., trastuzumab (Herceptin®) and other antibodies from Genentech).

antimetabolites (including, without limitation, folic acid antagonists (also referred to herein as antifolates), pyrimidine analogs, purine analogs and adenosine deaminase inhibitors): methotrexate (Rheumatrex®, Trexall®), 5-fluorouracil (Adrucil®, Efudex®, Fluoroplex®), floxuridine (FUDF®), cytarabine (Cytosar-U®, Tarabine PFS), 6-mercaptopurine (Puri-Nethol®)), 6-thioguanine (Thioguanine Tabloid®), fludarabine phosphate (Fludara®), pentostatin (Nipent®), pemetrexed (Alimta®), raltitrexed (Tomudex®), cladribine (Leustatin®), clofarabine (Clofarex®, Clolar®), mercaptopurine (Puri-Nethol®), capecitabine (Xeloda®), nelarabine (Arranon®), azacitidine (Vidaza®) and gemcitabine (Gemzar®). Preferred antimetabolites include, e.g., 5-fluorouracil (Adrucil®, Efudex®, Fluoroplex®), floxuridine (FUDF®), capecitabine (Xeloda®), pemetrexed (Alimta®), raltitrexed (Tomudex®) and gemcitabine (Gemzar®).

vinca alkaloids: vinblastine (Velban®, Velsar®), vincristine (Vincasar®, Oncovin®), vindesine (Eldisine®), vinorelbine (Navelbine®).

additional platinum-based agents: carboplatin (Paraplat®, Paraplatin®), cisplatin (Platinol®), oxaliplatin (Eloxatin®).

anthracyclines: daunorubicin (Cerubidine®, Rubidomycin®), doxorubicin (Adriamycin®), epirubicin (Ellence®), idarubicin (Idamycin®), mitoxantrone (Novantrone®), valrubicin (Valstar®). Preferred anthracyclines include daunorubicin (Cerubidine®, Rubidomycin®) and doxorubicin (Adriamycin®).

topoisomerase inhibitors: topotecan (Hycamtin®), irinotecan (Camptosar®), etoposide (Toposar®, VePesid®), teniposide (Vumon®), lamellarin D, SN-38, camptothecin (e.g., IT-101).

taxanes: paclitaxel (Taxol®), docetaxel (Taxotere®), larotaxel, cabazitaxel.

epothilones: ixabepilone, epothilone B, epothilone D, BMS310705, dehydelone, ZK-Epothilone (ZK-EPO).

antibiotics: actinomycin (Cosmegen®), bleomycin (Blenoxane®), hydroxyurea (Droxia®, Hydrea®), mitomycin (Mitozytrex®, Mutamycin®).

immunomodulators: lenalidomide (Revlimid®), thalidomide (Thalomid®).

immune cell antibodies: alemtuzamab (Campath®), gemtuzumab (Myelotarg®), rituximab (Rituxan®), tositumomab (Bexxar®).

interferons (e.g., IFN-alpha (Alferon®, Roferon-A®) Intron®-A) or IFN-gamma (Actimmune®))

interleukins: IL-1, IL-2 (Proleukin®), IL-24, IL-6 (Sigosix®), IL-12.

HSP90 inhibitors (e.g., geldanamycin or any of its derivatives). In certain embodiments, the HSP90 inhibitor is selected fromgeldanamycin, 17-alkylamino-17-desmethoxygeldanamycin (“17-AAG”) or 17-(2-dimethylaminoethyl)amino-17-desmethoxygeldanamycin (“17-DMAG”).

anti-androgens which include, without limitation nilutamide (Nilandron®) and bicalutamide (Caxodex®).

antiestrogens which include, without limitation tamoxifen (Nolvadex®), toremifene (Fareston®), letrozole (Femara®), testolactone (Teslac®), anastrozole (Arimidex®), bicalutamide (Casodex®), exemestane (Aromasin®), flutamide (Eulexin®), fulvestrant (Faslodex®), raloxifene (Evista®) Keoxifene®) and raloxifene hydrochloride.

anti-hypercalcaemia agents which include without limitation gallium (III) nitrate hydrate (Ganite®) and pamidronate disodium (Aredia®).

apoptosis inducers which include without limitation ethanol, 2-[[3-(2,3-dichlorophenoxy)propyl]amino]-(9Cl), gambogic acid, embelin and arsenic trioxide (Trisenox®).

Aurora kinase inhibitors which include without limitation binucleine 2.

Bruton's tyrosine kinase inhibitors which include without limitation terreic acid.

calcineurin inhibitors which include without limitation cypermethrin, deltamethrin, fenvalerate and tyrphostin 8.

CaM kinase II inhibitors which include without limitation 5-Isoquinolinesulfonic acid, 4-[{2S)-2-[(5-isoquinolinylsulfonyl)methylamino]-3-oxo-3-{4-phenyl-1-piperazinyl)propyl]phenyl ester and benzenesulfonamide.

CD45 tyrosine phosphatase inhibitors which include without limitation phosphonic acid.

CDC25 phosphatase inhibitors which include without limitation 1,4-naphthalene dione, 2,3-bis[(2-hydroxyethyl)thio]-(9Cl).

CHK kinase inhibitors which include without limitation debromohymenialdisine.

cyclooxygenase inhibitors which include without limitation 1H-indole-3-acetamide, 1-(4-chlorobenzoyl)-5-methoxy-2-methyl-N-(2-phenylethyl)-(9Cl), 5-alkyl substituted 2-arylaminophenylacetic acid and its derivatives (e.g., celecoxib (Celebrex®), rofecoxib (Vioxx®), etoricoxib (Arcoxia®), lumiracoxib (Prexige®), valdecoxib (Bextra®) or 5-alkyl-2-arylaminophenylacetic acid).

cRAF kinase inhibitors which include without limitation 3-(3,5-dibromo-4-hydroxybenzylidene)-5-iodo-1,3-dihydroindol-2-one and benzamide, 3-(dimethylamino)-N-[3-[(4-hydroxybenzoyl)amino]-4-methylphenyl]-(9Cl).

cyclin dependent kinase inhibitors which include without limitation olomoucine and its derivatives, purvalanol B, roascovitine (Seliciclib®), indirubin, kenpaullone, purvalanol A and indirubin-3′-monooxime.

cysteine protease inhibitors which include without limitation 4-morpholinecarboxamide, N-[1S)-3-fluoro-2-oxo-1-(2-phenylethyl)propyl]amino]-2-oxo-1-(phenylmethyl-1)ethyl]-(9Cl).

DNA intercalators which include without limitation plicamycin (Mithracin®) and daptomycin (Cubicin®).

DNA strand breakers which include without limitation bleomycin (Blenoxane®).

E3 ligase inhibitors which include without limitation N-((3,3,3-trifluoro-2-trifluoromethyl)propionyl)sulfanilamide.

EGF Pathway Inhibitors which include, without limitation tyrphostin 46, EKB-569, erlotinib (Tarceva®), gefitinib (Iressa®), lapatinib (Tykerb®) and those compounds that are generically and specifically disclosed in WO 97/02266, EP 0 564 409, WO 99/03854, EP 0 520 722, EP 0 566 226, EP 0 787 722, EP 0 837 063, U.S. Pat. No. 5,747,498, WO 98/10767, WO 97/30034, WO 97/49688, WO 97/38983 and WO 96/33980.

farnesyltransferase inhibitors which include without limitation A-hydroxyfarnesylphosphonic acid, butanoic acid, 2-[(2S)-2-[[(2S,3S)-2-[[(2R)-2-amino-3-mercaptopropyl]amino]-3-methylpent-yl]oxy]-1-oxo-3-phenylpropyl]amino-1-4-(methylsulfonyl)-1-methylethylester (2S)-(9Cl), and manumycin A.

Flk-1 kinase inhibitors which include without limitation 2-propenamide, 2-cyano-3-[4-hydroxy-3,5-bis(1-methylethyl)phenyl]-N-(3-phenylpropyl)-(2E-)-(9Cl).

glycogen synthase kinase-3 (GSK3) inhibitors which include without limitation indirubin-3′-monooxime.

histone deacetylase (HDAC) inhibitors which include without limitation suberoylanilide hydroxamic acid (SAHA), [4-(2-amino-phenylcarbamoyl)-benzyl]-carbamic acid pyridine-3-ylmethylester and its derivatives, butyric acid, pyroxamide, trichostatin A, oxamflatin, apicidin, depsipeptide, depudecin, trapoxin and compounds disclosed in WO 02/22577.

I-kappa B-alpha kinase inhibitors (IKK) which include without limitation 2-propenenitrile, 3-[(4-methylphenyl)sulfonyl]-(2E)-(9Cl).

imidazotetrazinones which include without limitation temozolomide (Methazolastone®, Temodar® and its derivatives (e.g., as disclosed generically and specifically in U.S. Pat. No. 5,260,291) and Mitozolomide.

insulin tyrosine kinase inhibitors which include without limitation hydroxyl-2-naphthalenylmethylphosphonic acid.

c-Jun-N-terminal kinase (JNK) inhibitors which include without limitation pyrazoleanthrone and epigallocatechin gallate.

mitogen-activated protein kinase (MAP) inhibitors which include without limitation benzenesulfonamide, N-[2-[[[3-(4-chlorophenyl)-2-propenyl]methyl]amino]methyl]phenyl]-N-(2-hydroxyethyl)-4-methoxy-(9Cl).

MDM2 inhibitors which include without limitation trans-4-iodo, 4′-boranyl-chalcone.

MEK inhibitors which include without limitation butanedinitrile, bis[amino[2-aminophenyl)thio]methylene]-(9Cl).

MMP inhibitors which include without limitation Actinonin, epigallocatechin gallate, collagen peptidomimetic and non-peptidomimetic inhibitors, tetracycline derivatives marimastat (Marimastat®), prinomastat, incyclinide (Metastat®), shark cartilage extract AE-941 (Neovastat®), Tanomastat, TAA211, MMI270B or AAJ996.

mTor inhibitors which include without limitation rapamycin (Rapamune®), and analogs and derivatives thereof, AP23573 (also known as ridaforolimus, deforolimus, or MK-8669), CCI-779 (also known as temsirolimus) (Torisel®) and SDZ-RAD.

NGFR tyrosine kinase inhibitors which include without limitation tyrphostin AG 879.

p38 MAP kinase inhibitors which include without limitation Phenol, 4-[4-(4-fluorophenyl)-5-(4-pyridinyl)-1H-imidazol-2-yl]-(9Cl), and benzamide, 3-(dimethylamino)-N-[3-[(4-hydroxylbenzoyl)amino]-4-methylphenyl]-(9Cl).

p56 tyrosine kinase inhibitors which include without limitation damnacanthal and tyrphostin 46.

PDGF pathway inhibitors which include without limitation tyrphostin AG 1296, tyrphostin 9,1,3-butadiene-1,1,3-tricarbonitrile, 2-amino-4-(1H-indol-5-yl)-(9Cl), imatinib (Gleevec®) and gefitinib (Iressa®) and those compounds generically and specifically disclosed in European Patent No. 0 564 409 and PCT Publication No. WO 99/03854.

phosphatidylinositol 3-kinase inhibitors which include without limitation wortmannin, and quercetin dihydrate.

phosphatase inhibitors which include without limitation cantharidic acid, cantharidin, and L-leucinamide.

protein phosphatase inhibitors which include without limitation cantharidic acid, cantharidin, L-P-bromotetramisole oxalate, 2(5H)-furanone, 4-hydroxy-5-(hydroxymethyl)-3-(1-oxohexadecyl)-(5R)-(9Cl) and benzylphosphonic acid.

PKC inhibitors which include without limitation 1-H-pyrollo-2,5-dione,3-1-[[3-(dimethylamino)propyl]-1H-indol-3-yl]-4-(1H-indol-3-yl)-(9Cl), Bisindolylmaleimide IX, Sphinogosine, staurosporine, and Hypericin.

PKC delta kinase inhibitors which include without limitation rottlerin.

polyamine synthesis inhibitors which include without limitation DMFO.

proteasome inhibitors which include, without limitation aclacinomycin A, gliotoxin and bortezomib (Velcade®).

PTP1B inhibitors which include without limitation L-leucinamide. protein tyrosine kinase inhibitors which include, without limitation tyrphostin Ag 216, tyrphostin Ag 1288, tyrphostin Ag 1295, geldanamycin, genistein and 7H-pyrollo[2,3-d]pyrimidine derivatives as generically and specifically described in PCT Publication No. WO 03/013541 and U.S. Publication No. 2008/0139587.

SRC family tyrosine kinase inhibitors which include without limitation PP1 and PP2.

Syk tyrosine kinase inhibitors which include without limitation piceatannol.

Janus (JAK-2 and/or JAK-3) tyrosine kinase inhibitors which include without limitation tyrphostin AG 490 and 2-naphthyl vinyl ketone.

retinoids which include without limitation isotretinoin (Accutane®, Amnesteem®, Cistane®, Claravis®, Sotret®) and tretinoin (Aberel®, Aknoten®, Avita®, Renova®, Retin-A®, Retin-A MICRO®, Vesanoid®).

RNA polymerase II elongation inhibitors which include without limitation 5,6-dichloro-1-beta-D-ribofuranosylbenzimidazole.

serine/Threonine kinase inhibitors which include without limitation 2-aminopurine.

sterol biosynthesis inhibitors which include without limitation squalene epoxidase and CYP2D6.

VEGF pathway inhibitors, which include without limitation anti-VEGF antibodies, e.g., bevacizumab, and small molecules, e.g., sunitinib (Sutent®), sorafinib (Nexavar®), ZD6474 (also known as vandetanib) (Zactima™), SU6668, CP-547632 and AZD2171 (also known as cediranib) (Recentin™).

Examples of chemotherapeutic agents are also described in the scientific and patent literature, see, e.g., Bulinski (1997) J. Cell Sci. 110:3055-3064; Panda (1997) Proc. Natl. Acad. Sci. USA 94:10560-10564; Muhlradt (1997) Cancer Res. 57:3344-3346; Nicolaou (1997) Nature 387:268-272; Vasquez (1997) Mol. Biol. Cell. 8:973-985; Panda (1996) J. Biol. Chem. 271:29807-29812.

In some embodiments, the copper chelator can be administered with the platinum-based therapeutic instead of administration of a platinum-based therapeutic alone, e.g., instead of a platinum-based therapeutic as a first line therapy or a second line therapy.

In embodiments, a hormone and/or steroid can be administered in combination with a copper chelator and platinum-based chemotherapeutic. Examples of hormones and steroids include: 17a-ethinylestradiol (Estinyl®, Ethinoral®, Feminone®, Orestralyn®), diethylstilbestrol (Acnestrol®, Cyren A®, Deladumone®, Diastyl®, Domestrol®, Estrobene®, Estrobene®, Estrosyn®, Fonatol®, Makarol®, Milestrol®, Milestrol®, Neo-Oestronol I®, Oestrogenine®, Oestromenin®, Oestromon®, Palestrol®, Stilbestrol®, Stilbetin®, Stilboestroform®, Stilboestrol®, Synestrin®, Synthoestrin®, Vagestrol®), testosterone (Delatestryl®, Testoderm®, Testolin®, Testostroval®, Testostroval-PA®, Testro AV)), prednisone (Delta-Dome®, Deltasone®, Liquid Pred®, Lisacort®, Meticorten®, Orasone®, Prednicen-M®, Sk-Prednisone®, Sterapred®), Fluoxymesterone (Android-F®, Halodrin®, Halotestin®, Ora-Testryl®, Ultandren®), dromostanolone propionate (Drolban®, Emdisterone®, Masterid®, Masteril®, Masteron®, Masterone®, Metholone®, Permastril®), testolactone (Teslac®), megestrolacetate (Magestin®, Maygace®, Megace®, Megeron®, Megestat®, Megestil®, Megestin®, Nia®, Niagestin®, Ovaban®, Ovarid®, Volidan®), methylprednisolone (Depo-Medrol®, Medlone 21®, Medrol®, Meprolone®, Metrocort®, Metypred®, Solu-Medrol®, Summicort®), methyl-testosterone (Android®, Testred®, Virilon®), prednisolone (Cortalone®, Delta-Cortef®, Hydeltra®, Hydeltrasol®, Meti-derm®, Prelone®), triamcinolone (Aristocort®), chlorotrianisene (Anisene®, Chlorotrisin®, Clorestrolo®, Clorotrisin®, Hormonisene®, Khlortrianizen®, Merbentul®, Metace®, Rianil®, Tace®, Tace-Fn®, Trianisestrol®), hydroxyprogesterone (Delalutin®, Gestiva™), aminoglutethimide (Cytadren®, Elipten®, Orimeten®), estramustine (Emcyt®), medroxyprogesteroneacetate (Provera®, Depo-Provera®), leuprolide (Lupron®, Viadur®), flutamide (Eulexin®), toremifene (Fareston®), and goserelin (Zoladex®).

In embodiments, the copper chelator and platinum-based chemotherapeutic may be administered in combination with an anti-microbial (e.g., leptomycin B).

In an embodiment, the copper chelator and platinum-based chemotherapeutic may be administered in combination with an agent or procedure to mitigate potential side effects from the agent compositions such as diarrhea, nausea and vomiting.

Diarrhea may be treated with antidiarrheal agents including, but not limited to opioids (e.g., codeine (Codicept®, Coducept®), oxicodeine, percocet, paregoric, tincture of opium, diphenoxylate (Lomotil®), diflenoxin), and loperamide (Imodium A-D®), bismuth subsalicylate, lanreotide, vapreotide (Sanvar®, Sanvar IR®), motiln antagonists, COX2 inhibitors (e.g., celecoxib (Celebrex®), glutamine (NutreStore®), thalidomide (Synovir®, Thalomid®), traditional antidiarrhea remedies (e.g., kaolin, pectin, berberine and muscarinic agents), octreotide and DPP-IV inhibitors.

DPP-IV inhibitors employed in the methods described herein are generically and specifically disclosed in PCT Publication Nos.: WO 98/19998, DE 196 16 486 A1, WO 00/34241 and WO 95/15309.

Nausea and vomiting may be treated with antiemetic agents such as dexamethasone (Aeroseb-Dex®, Alba-Dex®, Decaderm®, Decadrol®, Decadron®, Decasone®, Decaspray®, Deenar®, Deronil®, Dex-4®, Dexace®, Dexameth®, Dezone®, Gammacorten®, Hexadrol®, Maxidex®, Sk-Dexamethasone®), metoclopramide (Reglan®), diphenylhydramine (Benadryl®, SK-Diphenhydramine®), lorazepam (Ativan®), ondansetron (Zofran®), prochlorperazine (Bayer A 173®, Buccastem®, Capazine®, Combid®, Compazine®, Compro®, Emelent®, Emetiral®, Eskatrol®, Kronocin®, Meterazin®, Meterazin Maleate®, Meterazine®, Nipodal®, Novamin®, Pasotomin®, Phenotil®, Stemetil®, Stemzine®, Tementil®, Temetid®, Vertigon®), thiethylperazine (Norzine®, Torecan®), and dronabinol (Marinol®).

In some embodiments, the copper chelator and platinum-based chemotherapeutic may be administered in combination with an immunosuppressive agent. Immunosuppressive agents suitable for the combination include, but are not limited to natalizumab (Tysabri®), azathioprine (Imuran®), mitoxantrone (Novantrone®), mycophenolate mofetil (Cellcept®), cyclosporins (e.g., Cyclosporin A (Neoral®, Sandimmun®, Sandimmune®, SangCya®), calcineurin inhibitors (e.g., Tacrolimus (Prograf®, Protopic®), sirolimus (Rapamune®), everolimus (Afinitor®), cyclophosphamide (Cytoxan®, Neosar®), or methotrexate (Abitrexate®, Folex®, Methotrexate®, Mexate®)), fingolimod, mycophenolate mofetil (CellCept®), mycophenolic acid (Myfortic®), anti-CD3 antibody, anti-CD25 antibody (e.g., Basiliximab (Simulect®) or daclizumab (Zenapax®)), and anti-TNF.alpha. antibody (e.g., Infliximab (Remicade®) or adalimumab (Humira®)).

In some embodiments, a copper chelator and platinum-based chemotherapeutic are administered in combination with a CYP3A4 inhibitor (e.g., ketoconazole (Nizoral®, Xolegel®), itraconazole (Sporanox®), clarithromycin (Biaxin®), atazanavir (Reyataz®), nefazodone (Serzone®, Nefadar®), saquinavir (Invirase®), telithromycin (Ketek®), ritonavir (Norvir®), amprenavir (also known as Agenerase, a prodrug version is fosamprenavir (Lexiva®, Telzir®), indinavir (Crixivan®), nelfinavir (Viracept®), delavirdine (Rescriptor®) or voriconazole (Vfend®)).

When employing the methods or compositions, other agents used in the modulation of tumor growth or metastasis in a clinical setting, such as antiemetics, can also be administered as desired.

Exemplary agents that can be administered with a copper chelator and platinum-based chemotherapeutic include, e.g., when the platinum-based chemotherapeutic is cisplatin: pemetrexed (ALIMTA®), vinorelbine (Navelbine®), gemcitabine (Gemzar®) vinblastine (Velban®, Velsar®), dacarbazine (DTIC-Dome®) temozolomide (Methazolastone®, Temodar®), 5FU (Adrucil®, Efudex®, Fluoroplex®), cyclophosphamide (Cytoxan®, Neosar®, Endoxan®, Procytox®, Revimmune™), bleomycin (Blenoxane®), etoposide (Toposar®, VePesid®), ifosfamide (Mitoxana®), paclitaxel (Taxol®), methotrexate (Abitrexate®, Folex®, Methotrexate®, Mexate®, Rheumatrex®, Trexall®), doxorubicin (Adriamycin®), vincristine (Vincasar®, Oncovin®), mitomycin (Mitozytrex®, Mutamycin®), docetaxel (Taxotere®), vinorelbine (Navelbine®), and combinations of the above agents. The above agents may also be administered in conjunction with surgery and/or radiation.

When the platinum-based chemotherapeutic is carboplatin, exemplary agents that can be administered with the copper chelator and carboplatin include, e.g., irinotecan (Camptosar®), leucovorin (Wellcovorin®), 5FU (Adrucil®, Efudex®, Fluoroplex®), capecitabine (Xeloda®), bevacizumab (Avastin®), paclitaxel (Taxol®), cyclophosphamide (Cytoxan®, Neosar®, Endoxan®, Procytox®, Revimmune™), docetaxel (Taxotere®), gemcitabine (Gemzar®), etoposide (Toposar®, VePesid®), ifosfamide (Mitoxana®), vinorelbine (Navelbine®), doxorubicin (Adriamycin®), methotrexate (Abitrexate®, Folex®, Methotrexate®, Mexate®, Rheumatrex®, Trexall®), vincristine (Vincasar®, Oncovin®), and combinations of the above agents. The above agents may also be administered in conjunction with surgery and/or radiation.

When the platinum-based chemotherapeutic is oxaliplatin, exemplary agents that can be administered with the copper chelator and carboplatin include, e.g., leucovorin (Wellcovorin®), and 5FU (Adrucil®, Efudex®, Fluoroplex®), and combinations of the above agents. The above agents may also be administered in conjunction with surgery and/or radiation.

When formulating the pharmaceutical compositions described herein, the clinician may utilize preferred dosages as warranted by the condition of the subject being treated. For example, in one embodiment, a copper chelator and platinum-based chemotherapeutic may be administered at a dosing schedule described herein, e.g., once every one, two, three, four, five or six weeks.

Also, in general, a copper chelator, a platinum-based chemotherapeutic, and an optional additional chemotherapeutic agent(s) do not have to be administered in the same pharmaceutical composition, and may, because of different physical and chemical characteristics, have to be administered by different routes. For example, the copper chelator may be administered orally, the platinum-based chemotherapeutic may be administered intravenously, and the additional chemotherapeutic agent(s) may be administered orally or intravenously. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

The actual dosage of the copper chelator and platinum-based chemotherapeutic and/or any additional chemotherapeutic agent employed may be varied depending upon the requirements of the subject and the severity of the condition being treated. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached.

In some embodiments, when a copper chelator and platinum-based chemotherapeutic are administered in combination with one or more additional chemotherapeutic agents, the additional chemotherapeutic agent (or agents) is administered at a standard dose.

The particular choice of additional anti-proliferative cytotoxic agent(s) or radiation will depend upon the diagnosis of the attending physicians and their judgment of the condition of the subject and the appropriate treatment protocol.

If the copper chelator and platinum-based chemotherapeutic and the additional chemotherapeutic agent(s) and/or radiation are not administered simultaneously or essentially simultaneously, then the initial order of administration of the copper chelator and platinum-based chemotherapeutic, and the additional chemotherapeutic agent(s) and/or radiation, may be varied. Thus, for example, the copper chelator and platinum-based chemotherapeutic may be administered first followed by the administration of the additional chemotherapeutic agent(s) and/or radiation; or the additional chemotherapeutic agent(s) and/or radiation may be administered first followed by the administration of the copper chelator and platinum-based chemotherapeutic. This alternate administration may be repeated during a single treatment protocol. The determination of the order of administration, and the number of repetitions of administration of each therapeutic agent during a treatment protocol, is well within the knowledge of the skilled physician after evaluation of the disease being treated and the condition of the subject.

Thus, in accordance with experience and knowledge, the practicing physician can modify each protocol for the administration of a component (copper chelator and platinum-based chemotherapeutic, anti-neoplastic agent(s), or radiation) of the treatment according to the individual subject's needs, as the treatment proceeds.

The attending clinician, in judging whether treatment is effective at the dosage administered, will consider the general well-being of the subject as well as more definite signs such as relief of disease-related symptoms, inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

The following non-limiting Examples are intended to be purely illustrative, and show specific experiments that were carried out in accordance with the disclosure.

### EXAMPLES

### Materials & Methods

Bladder cell lines (5637, J82, RT4, MB49, MBT2, and NBT2) were kindly provided by Brant A. Inman, MD. J82, NBT2, MB49, and MBT2 cells were cultured in Dulbecco's modified Eagle's medium (DMEM) containing 10% fetal bovine serum (FBS) and 1% antibiotic-antimycotic (AA; Invitrogen). RT4 and 5637 cells were cultured in McCoy's 5A modified medium and RPMI-1640 medium, respectively, containing 10% FBS and 1% AA. The WT and Ctr1−/− MEF cell lines as well as the Ctr1 antibody were kindly provided by Dennis J. Thiele, PhD. MEF cells were cultured in DMEM containing 20% FBS, 1% L-Glutamine-Penicillin-Streptomycin (Sigma), 1% sodium pyruvate, 1% nonessential amino acids, 0.5 mg/L uridine (Sigma), and 55 μM 2-mercaptoethanol. All cells were maintained at 37° C. and at 5% CO₂.

### Example 1

### Ctr1 Expression in Bladder Cancer Cell Lines

Basal Ctr1 protein expression was assessed using Western blot in several different human (5637, J82, and RT4) and rodent (MB49, MBT2, and NBT2) bladder cancer cell lines. Briefly, cells were plated in 10-cm culture dishes and harvested approximately 48-72 hours after plating. Cells were lysed in 1 M Tris (pH7.5), 5 M NaCl, 100 mM EDTA, 100 mM EGTA, 10% Triton X-100, and protease inhibitor mixture (1:100). Samples were centrifuged at 12,000 rpm, and 100 μg of protein was separated using 4-20% SDS-PAGE, transferred to PVDF membrane and detected by chemiluminescence using anti-Ctr1 antibody (1:1000; kindly provided by Dennis J. Thiele, PhD) followed by anti-rabbit HRP antibody (1:2000; Bio-Rad). Sensitivity to cisplatin was assessed using a clonogenic survival assay. Briefly, a known density of cells was plated in 6-well plates. Approximately 18-24 hours after plating, cells were exposed to cisplatin (1, 5, 10, or 50 μM; Sigma) or saline for 2 hours. Following cisplatin exposure, media was removed, cells were washed with PBS, and fresh media was added. Cells were incubated until colonies (50+ cells) were detected (7-14 days). Colonies were then stained using crystal violet and counted.

Results are illustrated in FIG. 1. The data indicates that cells expressing higher basal levels of Ctr1 (5637 and MBT2) may be more sensitive to cisplatin.

### Example 2

### Sensitivity to Cisplatin in Ctr1−/− MEFs

Basal Ctr1 protein expression was assessed using western blot in WT and Ctr1−/− mouse embryonic fibroblasts (MEFs). Sensitivity to cisplatin was assessed using a clonogenic survival assay. Briefly, a known density of cells was plated in 6-well plates. Approximately 18-24 hours after plating, cells were exposed to cisplatin (1, 5, or 10 μM; APP Pharmaceuticals; 50 mg/50 mL) or saline for 2 hours. Following cisplatin exposure, media was removed, cells were washed with PBS, and fresh media was added. Cells were incubated until colonies (50+ cells) were detected (7-14 days). Colonies were then stained using crystal violet and counted.

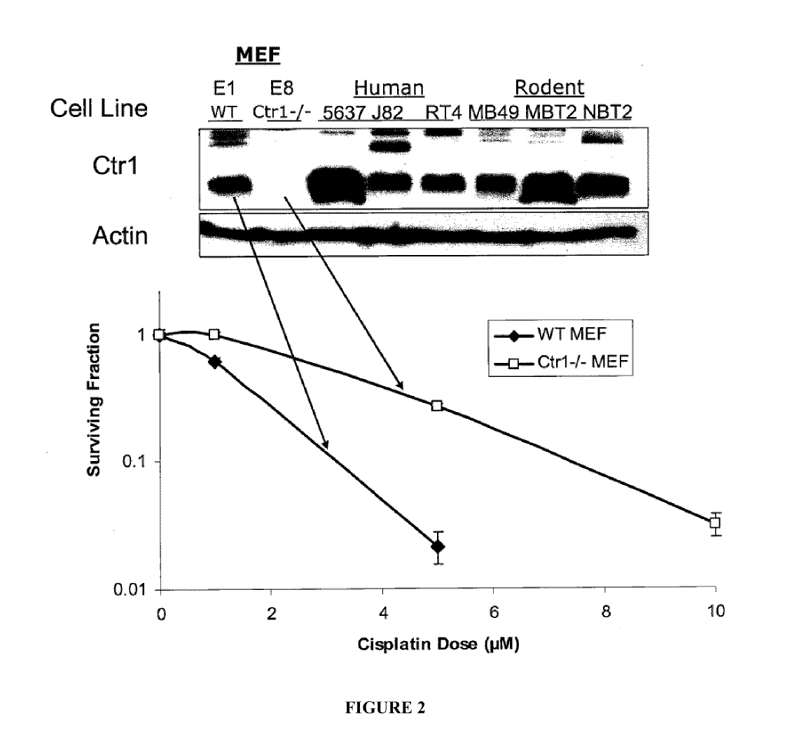

Results are illustrated in FIG. 2. The data indicates that cells expressing higher basal Ctr1 levels (WT) may be more sensitive to cisplatin, whereas cells that do not express Ctr1 are more resistant.

### Example 3

### Cisplatin Uptake/Accumulation in Ctr1−/− MEFs

WT and Ctr1−/− MEFs were plated into T175 flasks and treated with concurrent hyperthermia (42° C.) or normothermia (37° C.) and cisplatin (100 μg/mL; APP Pharmaceuticals; 50 mg/50 mL) for 1 hour. Following this 1 hour incubation, cells were incubated with cisplatin for an additional hour at 37° C. Cells were then washed twice with PBS and harvested into 1 mL of 3N HCl and 10% TCA. Samples were digested at 70° C. for 18 hours and centrifuged at 20,800×g for 5 minutes. The supernatants were collected, and intracellular platinum accumulation was measured using inductively coupled plasma atomic emission spectroscopy (ICP-AES) at the EPA, Chapel Hill, N.C.

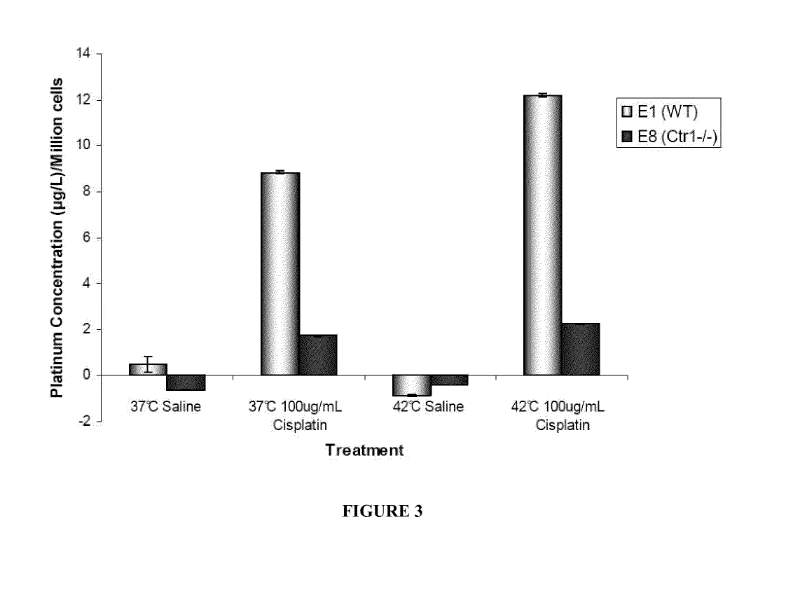

Results are illustrated in FIG. 3. Higher platinum levels were observed in WT cells compared to Ctr1−/− cells with and without hyperthermia. There was a further increase in platinum uptake in WT cells after hyperthermia that was not observed in Ctr1−/− cells.

### Example 4

### Platinum-DNA Adduct Formation in Ctr1−/− MEFs

WT and Ctr1−/− MEFs were plated on coverslips (80,000-90,000 cells) in 6-well plates. Approximately 24-48 hours after plating, cells were treated with or without concurrent mild hyperthermia (42° C.) or normothermia (37° C.) and 50 μg/mL cisplatin (APP Pharmaceuticals; 50 mg/50 mL) for 1 hour. Following this initial 1 hour treatment, cells were incubated for 3 additional hours at 37° C. Following treatment, platinum-DNA adducts were stained using immunofluorescence. Briefly, cells were washed with PBS and fixed with 2% paraformaldehyde. Cells were washed 3 additional times with PBS and permeabilized with 0.2% Triton X-100 for 5 minutes at 4° C. After blocking with 1% BSA for 30 minutes, cells were incubated with the primary monoclonal antibody for cisplatin-induced Pt-(GG) intrastrand adducts in DNA (0.5 μg/mL in 1% BSA; ONCOLYZE, Germany) overnight at 4° C. Cells were then washed with 1% BSA 3 times (10 minutes) and incubated with an Alexa 488 anti-rat antibody (1:1000 in 1% BSA; Jackson ImmunoResearch Laboratories) at 37° C. for 30 minutes. Following antibody incubation, coverslips were washed twice with PBS, incubated with Hoechst for 5 minutes, washed again with PBS, and mounted on slides. Cells were imaged using a Zeiss Axio Imager widefield fluorescence microscope (Carl Zeiss MicroImaging).

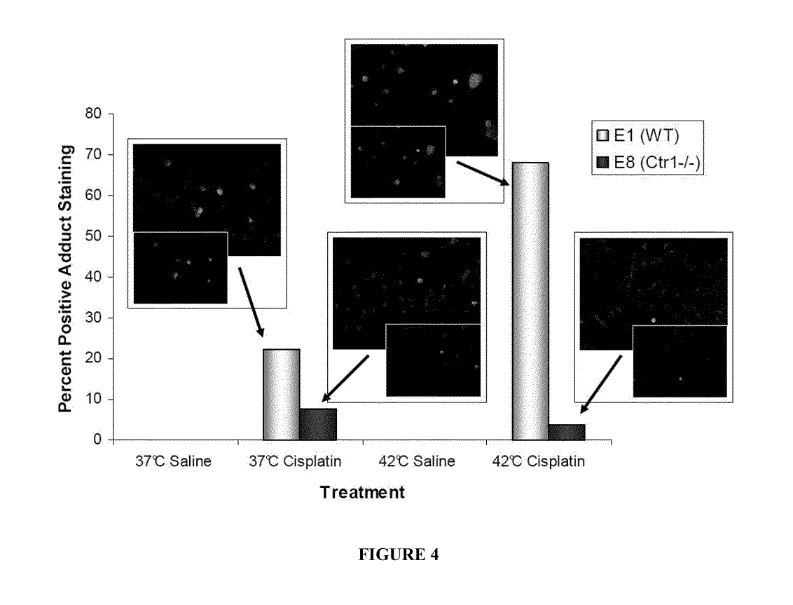

Results are illustrated in FIG. 4. Higher percentages of adduct-positive cells and adduct formation in WT were observed compared to Ctr1−/− cells, particularly following hyperthermia.

### Example 5

### Platinum-DNA Adduct Formation in Bladder Cancer Cells Following Ctr1 mRNA Knockdown

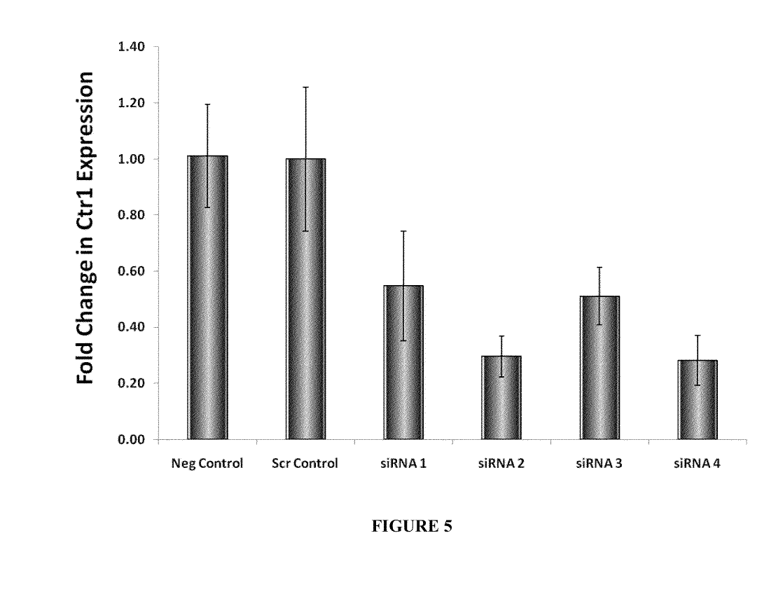

Ctr1 mRNA expression was assessed using RT-PCR 24 hours after transfection in 5637 human bladder cancer cells. This cell line was selected because of its relatively high basal Ctr1 expression levels (as confirmed by Western analysis). Briefly, 4×10⁵ cells were plated in 6-well plates. Approximately 24 hours after plating, cells were transfected with 10 nM scramble control or Ctr1 siRNA (QIAGEN) using the HiPerFect transfection reagent (QIAGEN). mRNA was harvested 24 hours after transfection using the RNeasy Mini Kit (QIAGEN). cDNA was synthesized using the iScript cDNA Synthesis Kit (Bio-Rad), and RT-PCR was conducted at the Duke IGSP Facility using the iTaq SYBR Green Supermix with ROX and previously published Ctr1 primers (Kitada et al. 2008 Cancer Chemother Pharmacol 62:577-584; forward: ACAAGTCAGCATTCGCTACAATTC (SEQ ID NO:1) and reverse: TTGCAGGAGGTGAGGAAAGC (SEQ ID NO:2) (5′-3′). Results are illustrated in FIG. 5.

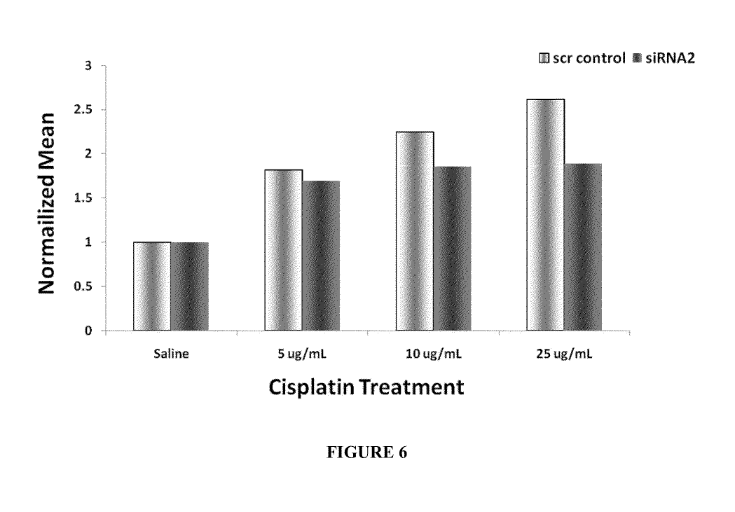

Platinum-DNA adduct formation was assessed using flow cytometry following a 4-hour incubation with cisplatin (or saline). Approximately 70 hours following transfection with Ctr1 siRNA2 (QIAGEN), 5637 cells were incubated with a range of cisplatin doses (5, 10, and 25 μg/mL) at 37° C. Cells were harvested and washed 3 times with PBS and fixed for 15 minutes with 2% paraformaldehyde at 4° C. Cells were washed 3 times with PBS and permeabalized with 0.25% Triton X-100 for 5 minutes at 4° C. Cells were incubated with the platinum-DNA adduct antibody (0.5 μg/mL in 1% BSA; ONCOLYZE) for 3 hours at room temperature. Following primary antibody incubation, cells were washed with 1% BSA and resuspended in the Alexa-488 anti-rat secondary antibody (1:500 in 1% BSA; Jackson ImmunoResearch Laboratories) for 30 minutes at room temperature. Cells were washed twice with PBS and resuspended in 500 μL of PBS for flow cytometry analysis conducted by the Duke University Cancer Center Flow Cytometry Shared Resource. Results are illustrated in FIG. 6.

### Example 6

### Sensitivity to Cisplatin in Cancer Cells Treated with a Copper Chelator

Cultures of cancer cell lines, e.g., bladder cancer cell lines such as human (5637, J82, and RT4) and rodent (MB49, MBT2, and NBT2) bladder cancer cells, will be incubated with a copper chelator such as D-penicillamine, trientine hydrochloride, ammonium tetrathiomolybdate, dimercaprol, sodium diethyldithiocarbamate or bathocuproine sulfonate, or with saline as a control. The cultures will be analyzed to confirm that incubation with the chelator does not inhibit cell proliferation. Additional parallel samples will be prepared that do not incorporate the incubation period with a copper chelator. The cells will subsequently be incubated with cisplatin, carboplatin or oxaliplatin, or with saline as a control. Sensitivity to cisplatin will be assessed using a clonogenic survival assay as described in Example 2. It is expected that the platinum compounds will show increased chemotherapeutic efficacy in the cell samples incubated with a copper chelator relative to the control cells, and the cells not incubated with a copper chelator.

### Example 7

### Cisplatin Uptake/Accumulation in Cancer Cells Treated with a Copper Chelator

Cultures of cancer cell lines, e.g., bladder cancer cell lines such as human (5637, J82, and RT4) and rodent (MB49, MBT2, and NBT2) bladder cancer cells, will be incubated with a copper chelator such as D-penicillamine, trientine hydrochloride, ammonium tetrathiomolybdate, dimercaprol, sodium diethyldithiocarbamate or bathocuproine sulfonate, or with saline as a control. The cultures will be analyzed to confirm that incubation with the chelator does not inhibit cell proliferation. Additional parallel samples will be prepared that do not incorporate the incubation period with a copper chelator. The cells will subsequently be incubated with cisplatin, carboplatin or oxaliplatin, or with saline as a control. Following treatment, intracellular platinum accumulation will be measured using ICP-AES. It is expected that cells incubated with a copper chelator will demonstrate enhanced accumulation of the platinum compounds relative to the control cells, and the cells not incubated with a copper chelator.

### Example 8

### Platinum-DNA Adduct Formation in Cancer Cells Treated with a Copper Chelator

Cultures of cancer cell lines, e.g., bladder cancer cell lines such as human (5637, J82, and RT4) and rodent (MB49, MBT2, and NBT2) bladder cancer cells, will be incubated with a copper chelator such as D-penicillamine, trientine hydrochloride, ammonium tetrathiomolybdate, dimercaprol, sodium diethyldithiocarbamate or bathocuproine sulfonate, or with saline as a control. The cultures will be analyzed to confirm that incubation with the chelator does not inhibit cell proliferation. Additional parallel samples will be prepared that do not incorporate the incubation period with a copper chelator. The cells will subsequently be incubated with cisplatin, carboplatin or oxaliplatin, or with saline as a control. Following treatment, platinum-DNA adducts will be stained using immunofluorescence. It is expected that cells incubated with a copper chelator will demonstrate enhanced platinum-DNA adduct formation relative to the control cells, and the cells not incubated with a copper chelator.

### Example 9

### Mouse Models

A pharmacokinetics study will be conducted prior to a tumor growth delay to confirm higher cisplatin concentration is being delivered to the tumor in the cisplatin+chelator group compared to control. We will use the Ctr1-expressing invasive human bladder transitional cell carcinoma line 5637 as a xenograft and/or human (J82, and RT4) or rodent (MB49, MBT2, and NBT2) bladder cancer cells. Tumor cells will be injected s.c. into the flank region of mice, and once tumors reach the appropriate size mice will be randomized into one of the treatment. Following cisplatin administration, mice will then be sacrificed at the time points from 15 minutes out to 48 hours. These time points were derived from several free cisplatin pharmacokinetic studies in mice. Blood as well as tumor, liver, spleen, kidneys, lungs, leg muscle, heart, brain, tumor, bone marrow from femurs, and colon tissues will be collected for cisplatin measurements. Platinum will be measured in plasma and tissues with ICP-MS, and adduct formation will be measured using IF staining of tumor sections.

Prior to conducting the pharmacokinetic study, we will treat tumor-bearing mice with copper chelator and determine the time required to significantly reduce blood copper concentration. We will also extract tumor tissue over a series of time points during chelation and measure Ctr1 levels. This time series will be used to decide the appropriate time (maximal Ctr1 expression) to treat mice with cisplatin in the pharmacokinetic and tumor growth delay studies.

Following analysis of the pharmacokinetic study, we will transition to a tumor growth delay study. A similar treatment protocol will be used as described above. Once tumors reach the appropriate size, mice will be randomized into one of the treatment groups. Chelators will be given orally prior to cisplatin treatment, and cisplatin will be administered as described above. The proper control groups will be used to compare the cisplatin+chelator group.

All patents, publications and references cited herein are hereby fully incorporated by reference. In case of conflict between the present disclosure and incorporated patents, publications and references, the present disclosure should control.

1. A method of reducing the proliferation of a cancer cell, comprising contacting the cancer cell with a copper chelator and a platinum-based chemotherapeutic.

2. The method of claim 1, wherein the cancer cell is a bladder, testicular, ovarian, head and neck, cervical, lung, Wilms' tumor, brain tumor, neuroblastoma, retinoblastoma, mesothelioma, esophageal or colorectal cancer cell.

3. The method of claim 1, wherein the copper chelator is penicillamine, bathocuproine sulfonate, sodium diethyldithiocarbamate, trientine hydrochloride, or dimercaprol, or any combination thereof.

4. The method of claim 1, wherein the copper chelator is penicillamine or trientine hydrochloride.

5. The method of claim 1, wherein the platinum-based therapeutic is selected from cisplatin, carboplatin and oxaliplatin.

6. The method of claim 1, wherein the cancer cell is contacted with the copper chelator and the platinum-based chemotherapeutic in vitro, in vivo or ex vivo.

7. The method of claim 1, wherein the cancer cell is first contacted with the copper chelator, and subsequently contacted with the platinum-based chemotherapeutic.

8. The method of claim 1, wherein the cancer cell is first contacted with the platinum-based chemotherapeutic, and subsequently contacted with the copper chelator.

9. The method of claim 1, wherein the cancer cell is simultaneously contacted with the copper chelator and the platinum-based chemotherapeutic.

10. The method of claim 1, further comprising contacting the cell with an additional chemotherapeutic agent.

11. A method of treating cancer in a subject in need of treatment, comprising administering to the subject a platinum-based chemotherapeutic and a copper chelator, in amounts effective to treat the cancer.

12. The method of claim 10, wherein the cancer is a bladder cancer, testicular cancer, ovarian cancer, head and neck cancer, cervical cancer, lung cancer, Wilms' tumor, brain tumor, neuroblastoma, retinoblastoma, mesothelioma, esophageal cancer or colorectal cancer.

13. The method of claim 10, wherein the copper chelator is penicillamine, bathocuproine sulfonate, sodium diethyldithiocarbamate, trientine hydrochloride, or dimercaprol, or any combination thereof.

14. The method of claim 10, wherein the copper chelator is penicillamine or trientine hydrochloride.

15. The method of claim 10, wherein the platinum-based therapeutic is cisplatin, carboplatin, or oxaliplatin, or any combination thereof.

16. The method of claim 10, wherein the platinum-based chemotherapeutic and the copper chelator are each independently administered parenterally or orally.

17. The method of claim 10, wherein the platinum-based chemotherapeutic is administered intravenously and the copper chelator is administered orally.

18. The method of claim 10, wherein the copper chelator is administered to the subject first, followed by subsequent administration of the platinum-based chemotherapeutic.

19. The method of claim 10, further comprising administering an additional chemotherapeutic agent to the subject.


SEQUENCE LISTING (2 sequences)

SEQ ID NO: 1 - DNA, 24 bp, Artificial Sequence
  Synthetic
acaagtcagcattcgctacaattc

SEQ ID NO: 2 - DNA, 20 bp, Artificial Sequence
  Synthetic
ttgcaggaggtgaggaaagc